(19) United States
(12) Patent Application Publication
(10) Pub. No.: US 2005/0271594 A1
(43) Pub. Date: Dec. 8, 2005

(54) Abuse resistent pharmaceutical composition

(76) Inventor: Pieter J. Groenewoud, Powder Springs, GA (US)

Correspondence Address: BRYAN W. BOCKHOP, ESQ., 2375 MOSSY BRANCH DR., SNELLVILLE, GA, 30078, US

(21) Appl. No.: 10/861,147

(22) Filed: Jun. 4, 2004

(51) Int. Cl. A61K 9/48; A61K 49/00

(52) U.S. Cl. 424/10.1; 424/451

(57) ABSTRACT

An abuse-resistant pharmaceutical composition includes an oily substance, at least one active ingredient having abuse potential and a capsule. The active ingredient is mixed in the oily substance. The oily substance and the active ingredient are placed in the capsule. In one embodiment the active ingredient includes oxicodone.

### BACKGROUND OF THE INVENTION

1. Field of the Invention

The invention relates to pharmaceutical compositions, and more specifically, to a pharmaceutical composition formulated to discourage abuse.

2. Description of the Related Art

Certain pharmaceutical compositions, including those subject to abuse, are available in sustained release formulations. Typical sustained release formulations have granules of the composition that are coated with a material that requires a predetermined time after ingestion prior to the release of the composition. For example, some granules of a given composition may be uncoated, some of the granules may be coated with a coating that takes three hours to dissolve and other granules may be coated with a coating that takes six hours to dissolve. Thus, there is an immediate release of the composition, followed by subsequent releases after three hours and six hours from ingestion.

Certain compositions, such as narcotics and stimulants are subject to abuse. In the sustained release form, some abusers crush the coated granules so as to gain immediate release of an entire dose. To speed up the effect, abusers often inhale or “snort” the crushed granules. Another method of abuse involves crushing the granules and then leaching out the active ingredient with vinegar to produce an injectable form of the narcotic.

Therefore, there is a need for a pharmaceutical formulation that inhibits abuse.

There is also a need for a pharmaceutical formulation that reduces the effects of an excessive dosage.

### SUMMARY OF THE INVENTION

In one aspect, the invention an abuse-resistant pharmaceutical composition that includes an oily substance, at least one active ingredient having abuse potential and a capsule. The active ingredient is mixed in the oily substance. The oily substance and the active ingredient are placed in the capsule. In one illustrative embodiment the active ingredient includes oxicodone.

In another aspect, the invention is an abuse-resistant pharmaceutical composition that includes an active ingredient in a dose of the pharmaceutical composition. A sub-therapeutic amount of an emetic is also included in the dose of the pharmaceutical composition. The emetic is in sufficient amount in the pharmaceutical composition so that if an undesirable amount of the active ingredient is ingested through the taking of multiple doses of the pharmaceutical composition, then a sufficient amount of emetic will also be ingested so as to be effective to induce vomiting.

In another aspect, the invention is a method for reducing abuse of a pharmaceutical composition subject to abuse. A pharmaceutically effective amount of an active ingredient in a sustained release formulation is mixed with an oily substance, thereby forming a pharmaceutical composition. The pharmaceutical composition is placed in a capsule and the capsule is sealed.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE DRAWINGS

FIG. 1 is a plan view of one illustrative embodiment of the invention.

FIG. 2 is a second plan view of an illustrative embodiment of the invention.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Also, as used herein, “timed release,” “sustained release,” “modified release” and “extended release” are used interchangeably and include compositions in which release of an active ingredient occurs not through a substantially immediate release episode, but occurs in one or more release episodes over a predetermined amount of time.

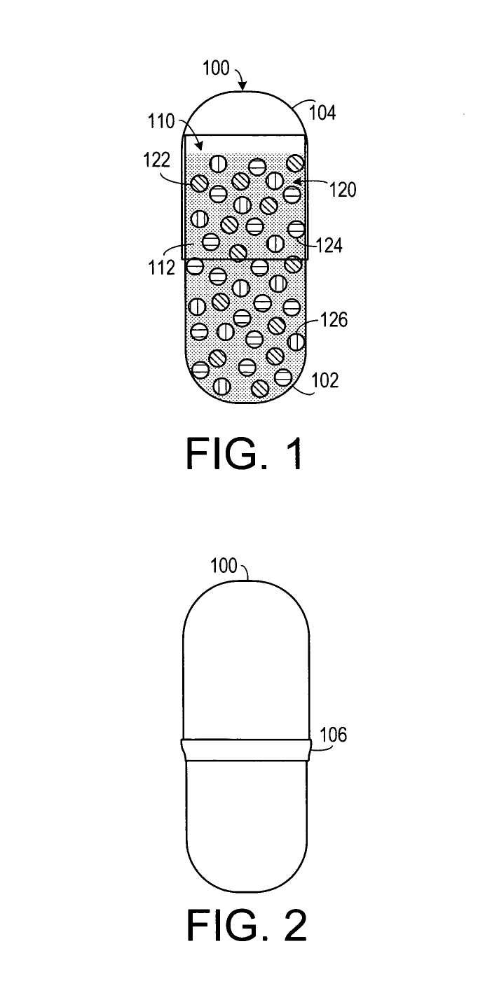

One embodiment of the invention, as shown in FIGS. 1-2, includes a capsule 100 into which is place an abuse-resistant pharmaceutical composition 110. The pharmaceutical composition 110 includes an oily substance carrier 112 that suspends various granules 120 of an active ingredient. In a sustained release formulation, the granules 120 could, for example, include relatively immediate release granules 122, relatively intermediate release granules 124 and relatively long term release granules 126.

The capsule 100 could include a body shell 102 and a dome shell 104, in a hard gelatin capsule embodiment. A soft gel-cap, of the type known to the art, could also be employed. A sealant 106 may also be applied to the capsule 100 to seal the body shell 102 to the dome shell 104. The technology for making liquid filled capsules is known in the art and information and equipment for such may be obtained from one of several companies, including, for example, Shionogi Qualicaps, Inc, 6505 Franz Warner Parkway, Whitsett, N.C. 27377-9215 (tel. 366-499-3900).

The active ingredient could be a narcotic, such as oxicodone or codeine, another analgesic, a stimulant, such as an amphetamine or methamphetamine, or any of many other pharmaceutical ingredients that are subject to, or otherwise have a potential for abuse. While the embodiment shown includes sustained release granules suspended in the oily substance, the active ingredient could also be dissolved in the oily substance without departing from the scope of the invention. Also, the active ingredient could be an immediate release formulation.

The oily substance could one of many oily substances, such as vegetable oils or synthetic oils, including: lipophilic liquids; hydrogenated oils; medium chain triglycerides; macrogols (polyethylene glycol); macrogol glycerides; and glyceryl esters of fatty acids. Suitable lipophilic liquids could include natural vegetable oils (including: arachis (groundnut), castor, cottonseed, maize (corn), olive, soybean, sunflower); propylene glycol laurate (Lauroglycol FCC); Glyceryl monolineate (Maisine 35-1); and Glyceryl monooleate (Peceol). Suitable hydrogenated oils could include; arachis (groundnut); castor; cottonseed; and soybean oils. Some suitable medium chain triglycerides could include the following caprylic/capric triglycerides: Bergabest; Captex 300 & 350; Labrafac cc; Miglyol 810 & 812; Myritol; Neobee M5; Nesatol; and Waglinol 3/9280. Some suitable macrogols (Polyethylene glycol) could include: Carbo wax; Lipoxol; Lutrol E; Pluriol E. Some suitable macrogol glycerides could include: Oleoyl-6 (Labrafil M 1944 CS); Linoleoyl-6 (Labrafil M2125 CS); Caprylocaaproyl (Labrasol); Lauroyl-32 (Gelucire 44/14); and Stearoyl-32 (Gelucire 50/13). Some suitable glyceryl esters of fatty acids could include Gelucire 33/01, 39/01, 43/01. It is understood that many other oily substances may be employed with the invention without departing from the scope of the claims below. It is also understood that “oily substance,” as applied to the claims, includes any liquid or gel-type substance that has a viscosity greater than water.

An emulsifier, such as a solubilizer or a surfactant may be added to create an emulsion (such as a cloudy emulsion) with the oily substance when a non-covalent liquid (such as vinegar) is added to the composition. The emulsifier would render an undesirable substance when an abuser attempts to leach out the active ingredient with vinegar, or other such liquids. One illustrative example of a suitable emulsifier includes a tween emulsifier, such as tween 20 or tween 80, or a span emulsifier. Examples of other suitable emulsifiers include solubilizers or surfactants, which may include: diethylene glycol monoethyl ether (Transcutol HP); Glyceryl monostearate (Imwitor); Polyglycerol-6 dioleate (Plurol oleique CC497); and Polyoxyethylene castor oil derivatives (Cremophor RH4O).

A viscosity enhancer may be mixed with the oily substance to increase the viscosity of the pharmaceutical composition, thereby making it harder to draw the pharmaceutical composition into a syringe. The viscosity enhancer could include aluminum stearate, a colloidal silicon dioxide Aerosil; or Cab-C-S/I; Wacker HDK. A temperature sensitive viscosity enhancer, such as a gellan gum (e.g., Kelcogel, Kelcogel F, Kelcogel LT 100 and K7B518, which are available from CP Kelco, 8355 Aero Drive, San Diego, Calif., 92123) could also be used according to the invention. Such a viscosity enhancer becomes highly viscous when heated, such as when an abuser is attempting to melt a pharmaceutical.

In one embodiment of the invention, a sub-therapeutic amount of an emetic is added to the dose of the pharmaceutical composition. The emetic is in sufficient amount in the pharmaceutical composition so that when a single dose of the pharmaceutical composition is ingested, the emetic has little effect. However, if an undesirable amount of the active ingredient is ingested through the taking of multiple doses of the pharmaceutical composition, then an amount of emetic will also be ingested that will be sufficient to induce vomiting. This aspect has the advantage of inhibiting intentional abuse and it also provides a safeguard against accidental overdose.

The above described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

1. An abuse-resistant pharmaceutical composition, comprising:
a. an oily substance;
b. at least one active ingredient having abuse potential mixed in the oily substance; and
c. a capsule into which the oily substance and the at least one active ingredient are placed.

2. The abuse-resistant pharmaceutical composition of claim 1, wherein the active ingredient comprises a narcotic.

3. The abuse-resistant pharmaceutical composition of claim 2, wherein the narcotic comprises oxicodone.

4. The abuse-resistant pharmaceutical composition of claim 1, wherein the active ingredient comprises a stimulant.

5. The abuse-resistant pharmaceutical composition of claim 1, wherein the oily substance is selected from the list consisting essentially of: lipophilic liquids, hydrogenated oils, medium chain triglycerides, macrogols, and glyceryl esters of fatty acids.

6. The abuse-resistant pharmaceutical composition of claim 1, further comprising an emulsifier mixed with the oily substance, the emulsifier capable of forming a cloudy emulsion when a non-covalent liquid is added to the pharmaceutical composition.

7. The abuse-resistant pharmaceutical composition of claim 6, wherein the emulsifier comprises a tween span emulsifier.

8. The abuse-resistant pharmaceutical composition of claim 7, wherein the tween span emulsifier comprises tween 20.

9. The abuse-resistant pharmaceutical composition of claim 7, wherein the tween span emulsifier comprises tween 80.

10. The abuse-resistant pharmaceutical composition of claim 6, wherein the emulsifier comprises a solubilizer.

11. The abuse-resistant pharmaceutical composition of claim 6, wherein the emulsifier comprises a surfactant.

12. The abuse-resistant pharmaceutical composition of claim 1, further comprising a viscosity enhancer mixed with the oily substance, the viscosity agent capable of increasing a viscosity of the pharmaceutical composition.

13. The abuse-resistant pharmaceutical composition of claim 12, wherein the viscosity enhancer comprises aluminum stearate.

14. The abuse-resistant pharmaceutical composition of claim 12, wherein the viscosity enhancer comprises a temperature sensitive viscosity enhancer.

15. The abuse-resistant pharmaceutical composition of claim 1, wherein the active ingredient comprises a plurality of sustained release granules that are suspended in the oily substance.

16. The abuse-resistant pharmaceutical composition of claim 1, wherein the active ingredient is dissolved in the oily substance.

17. The abuse-resistant pharmaceutical composition of claim 1, wherein the oily substance comprises a synthetic oil.

18. The abuse-resistant pharmaceutical composition of claim 1, wherein the oily substance comprises a vegetable oil.

19. The abuse-resistant pharmaceutical composition of claim 1, wherein the capsule comprises a soft gel cap.

20. The abuse-resistant pharmaceutical composition of claim 1, wherein the capsule comprises a hard gel cap.

21. The abuse-resistant pharmaceutical composition of claim 1, further comprising a sub-therapeutic amount of an emetic in the dose of the pharmaceutical composition, the emetic in sufficient amount in the pharmaceutical composition so that if an undesirable amount of the active ingredient is ingested through the taking of multiple doses of the pharmaceutical composition, then a sufficient amount of emetic will also be ingested so as to be effective to induce vomiting.

22. An abuse-resistant pharmaceutical composition, comprising:
a. an oily substance;
b. a pharmaceutically effective amount of oxicodone mixed in the oily substance; and
c. a capsule into which the oily substance and the oxicodone are placed.

23. The abuse-resistant pharmaceutical composition of claim 22, wherein the oxicodone comprises a sustained release formulation.

24. The abuse-resistant pharmaceutical composition of claim 22, wherein the oxicodone is suspended in the oily substance.

25. An abuse-resistant pharmaceutical composition, comprising:
a. an active ingredient in a dose of the pharmaceutical composition;
b. a sub-therapeutic amount of an emetic in the dose of the pharmaceutical composition, the emetic in sufficient amount in the pharmaceutical composition so that if an undesirable amount of the active ingredient is ingested through the taking of multiple doses of the pharmaceutical composition, then a sufficient amount of emetic will also be ingested so as to be effective to induce vomiting.

26. A method for reducing abuse of a pharmaceutical composition subject to abuse, comprising the steps of:
a. mixing a pharmaceutically effective amount of an active ingredient in a sustained release formulation with an oily substance, thereby forming a pharmaceutical composition;
b. placing the pharmaceutical composition in a capsule; and
c. sealing the capsule.

27. The method of claim 26, further comprising the step of adding an emulsifier to the pharmaceutical composition, wherein the emulsifier is capable of forming an emulsion with the oily substance when a non-covalent liquid is added to the pharmaceutical composition.

28. The method of claim 26, further comprising the steps of adding a viscosity agent to the pharmaceutical composition, wherein the viscosity agent capable of increasing a viscosity of the pharmaceutical composition.
